(19) United States
(12) Patent Application Publication
(10) Pub. No.: US 2025/0276110 A1
(43) Pub. Date: Sep. 4, 2025

(54) MENISCUS REPAIR IMPLANT AND METHOD OF USE

(71) Applicants: Smith & Nephew, Inc., Memphis, TN (US); Smith & Nephew Orthopaedics AG, Zug (CH); Smith & Nephew Asia Pacific Pte. Limited

(72) Inventors: Allison M. Stauffer, Brighton, MA (US); Matthew D. Cunningham, Lakeville, MA (US); Nehal N. Patel, Boston, MA (US)

(73) Assignees: Smith & Nephew, Inc., Memphis, TN (US); Smith & Nephew Orthopaedics AG, Zug (CH); Smith & Nephew Asia Pacific Pte. Limited, (omitted)

(21) Appl. No.: 19/212,086

(22) Filed: May 19, 2025

Related U.S. Application Data
(63) Continuation of application No. PCT/US2023/080530, filed on Nov. 20, 2023.
(60) Provisional application No. 63/427,287, filed on Nov. 22, 2022.

(51) Int. Cl. A61L 27/24; A61L 27/50

(52) U.S. Cl. CPC A61L 27/24; A61L 27/50; A61L 2430/06

(57) ABSTRACT

A collagen scaffold may be used in repairing a meniscal tear within a patient's meniscus. A collagen scaffold may be secured relative to a delivery device. The collagen scaffold may be placed within the meniscal tear and may be sutured in position. The sutures may pass through the collagen scaffold. The collagen fibers of the collagen scaffold may be substantially aligned with collagen fibers of the meniscus. In some cases, the collagen scaffold may be delivered to the meniscal tear using a securement member that includes forks that are adapted to extend into the collagen scaffold prior to folding and then rolling up the collagen scaffold and securement member to create a scaffold assembly that can be disposed within a delivery device.

### CROSS REFERENCE TO RELATED APPLICATIONS

This application is a continuation of International Application No. PCT/US2023/080530, filed on Nov. 20, 2023, which claims the benefit of U.S. Patent Application Ser. No. 63/427,287 filed on Nov. 22, 2022, the disclosures of which are incorporated herein by reference.

### TECHNICAL FIELD

The present disclosure pertains generally, but not by way of limitation, to orthopedic implants and methods of treatment. More particularly, the present disclosure relates to a meniscus repair implant, such as one that is engineered for use within portions of the meniscus that have little to no native blood flow

### BACKGROUND

The meniscus is part of the knee, and is a C-shaped piece of tough, rubbery cartilage that is made out of collagen and that acts as a shock absorber between the tibia and the femur. The meniscus includes a medial meniscus that is located on the inside of the knee and a lateral meniscus that sits on the outside of the knee. Meniscus tears are a common knee injury for athletes and non-athletes alike, and can occur when a patient twists or turns their upper leg while their foot is planted and their knee is bent. Treatment for meniscal tears can include simply cutting out the damaged portion of the meniscus. An advantage of this treatment is that the patient typically walks same day after surgery. A disadvantage of this treatment is that the patient ends up with less meniscal material, which does not replace itself. Another treatment for meniscus tears involves repairing the tear, which can be complicated by a relative lack of blood flow within the meniscus. An outer third of the meniscus is the “red” zone, which is vascularized and thus has the best healing potential. A middle third of the meniscus is the “red-white” zone, which has some blood flow. The inner third of the meniscus is the “white” zone, which lacks blood flow. Repairs to the “red-white” zone or the “red” zone may have poor healing results due to the lack of blood flow. Accordingly, there is an ongoing need to provide methods and materials for treating injuries to the meniscus.

### BRIEF SUMMARY

This disclosure provides design, material, manufacturing method, and use alternatives for implants used for soft tissue repairs such as but not limited to meniscus repairs. An implant may include a collagen scaffold. An example may be found in a method for repairing a meniscal tear within a patient's meniscus. The method includes securing a meniscal implant relative to a delivery device to form a scaffold delivery assembly and delivering the scaffold delivery assembly to a position proximate the meniscal tear. The meniscal implant is inserted into the meniscal tear and is held within the meniscal tear by placing one or more fasteners extending across the meniscal tear.

Alternatively or additionally, the method may further include separating the meniscal implant from the delivery device after inserting the meniscal implant into the meniscal tear.

Alternatively or additionally, the method may further include intra-operatively cutting the meniscal implant to an appropriate size prior to securing the meniscal implant to the delivery device.

Alternatively or additionally, the one or more fasteners may include one or more sutures.

Alternatively or additionally, the one or more sutures may pass through the meniscal implant.

Alternatively or additionally, the delivery device may include an elongate member, a cross member including a first cross member segment extending laterally from the elongate member in a first direction and a second cross member segment extending laterally from the elongate member in a second direction, and a plurality of fork members extending from the cross member, the plurality of fork members extending in a plane transverse to a plane extending through the elongate member and the cross member.

Alternatively or additionally, the method may further include intra-operatively imparting a bend to an elongate shaft of the delivery device prior to delivering the scaffold delivery assembly to the position proximate the meniscal tear.

Alternatively or additionally, the step of inserting the meniscal implant into the meniscal tear may include substantially aligning collagen fibers of the meniscal implant with collagen fibers of the meniscus.

Another example may be found in a method for delivering a treatment scaffold to a meniscal tear within a patient's meniscus. The method includes securing a treatment scaffold to a securement member adapted to releasably hold the treatment scaffold and loading the treatment scaffold and securement member into a delivery device. The treatment scaffold is deployed within the meniscal tear with the delivery device. The treatment scaffold is held within the meniscal tear by placing one or more sutures across the meniscal tear.

Alternatively or additionally, the delivery device may include an elongate shaft, and the method may further include bending the elongate shaft in order to align the treatment scaffold and securement member with the meniscal tear.

Alternatively or additionally, the delivery device may further include a handle, an actuatable trigger slidingly disposed within the handle, and a sheath adapted to fit over the securement member, the sheath operably coupled with the actuator trigger such that moving the trigger proximally relative to the handle withdraws the sheath from the securement member.

Alternatively or additionally, the delivery device may further include a cannula extending over the delivery device, the cannula removable before deploying the treatment scaffold.

Alternatively or additionally, the method may further include intra-operatively cutting the treatment scaffold to an appropriate size prior to securing the treatment scaffold to the securement member.

Alternatively or additionally, the method may further include selecting an appropriately sized treatment scaffold out of a plurality of differently sized treatment scaffolds.

Alternatively or additionally, the securement member may include an elongate member, a cross member extending laterally from the elongate member, and a plurality of fork members extending from the cross member, the plurality of fork members extending in a plane transverse to a plane extending through the elongate member and the cross member.

Alternatively or additionally, the step of deploying the treatment scaffold within the meniscal tear may include substantially aligning collagen fibers of the treatment scaffold with collagen fibers of the meniscus.

Alternatively or additionally, the one or more sutures may extend through the treatment implant.

Another example may be found in a device adapted for securing a meniscal implant for delivery to a meniscal tear. The device includes an elongate member, a cross member including a first cross member segment extending laterally from the elongate member in a first direction and a second cross member segment extending laterally from the elongate member in a second direction, and a plurality of fork members extending from the cross member, the plurality of fork members extending in a plane transverse to a plane extending through the elongate member and the cross member, at least some of the plurality of fork members adapted to extend into a meniscal implant. The first cross member segment and the second cross member segment are each adapted to fold over on themselves.

Alternatively or additionally, the cross member and the plurality of fork members may each include Nitinol.

Alternatively or additionally, the device may be constructed by processing a flat strip of Nitinol to form a blank including the elongate member, the cross member and the plurality of fork members, the blank extending within a single plane. The blank is shape set, with the plurality of fork members extending in the plane transverse to the plane extending through the elongate member and the cross member. Shape setting the blank further includes providing the delivery device with a remembered shape in which the first cross member segment extends laterally from the elongate member in a first direction, the second cross member segment extends laterally from the elongate member in a second direction, and the plurality of fork members extends in a plane transverse to a plane extending through the elongate member and the cross member.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIGS. 1A and 1B are perspective views of illustrative collagen scaffolds;

FIG. 2 is a side view of an illustrative blank that may be used to form an illustrative securement member;

FIG. 3 is a perspective view of an illustrative securement member formed from the blank of FIG. 2;

FIGS. 4 through 6 are perspective views of a process of forming an illustrative scaffold assembly; and

FIGS. 7 through 14 are perspective views of a process of implanting a treatment scaffold within a meniscal tear.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the disclosure.

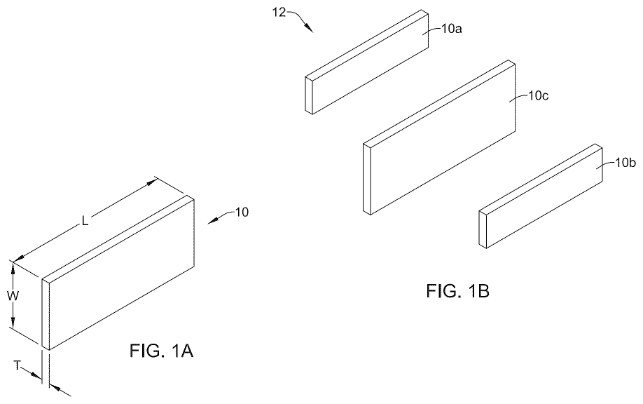

Meniscal implants such as treatment scaffolds may be provided in a variety of sizes. FIG. 1A shows an example of an illustrative treatment scaffold 10 having a length L, a width W and a thickness T. In some instances, L may vary from 10 to 40 millimeters in length, W may vary from 3 to 10 millimeters in width and T may vary from 1 to 5 millimeters thick. In some cases, the treatment scaffold 10 may be manufactured having particular dimensions, and prior to use, the physician or other professional may trim the treatment scaffold 10 in one or more dimensions in order to make the treatment scaffold 10 be an appropriate size for repairing a particular meniscal tear for a particular patient.

In some instances, the treatment scaffold 10 may be formed of a variety of different types of fibers. In some cases, the treatment scaffold 10 may include collagen fibers, particularly since the meniscus is largely formed of collagen and water. Some estimates place the meniscus as being about 72% water and about 22 percent collagen, with minor amounts of other materials. The treatment scaffold 10 may include collagen fibers that are loosely aligned along a long axis of the treatment scaffold 10. This may be beneficial, as the collagen fibers forming the C-shaped meniscus are themselves loosely aligned around the “C” shape. As a result of aligning the treatment scaffold 10 with its long dimension extending along the arcuate direction of the “C” shape, the collagen fibers within the treatment scaffold 10 may be oriented generally parallel to the collagen fibers forming the meniscus such that the collagen fibers within the treatment scaffold 10 are generally aligned with the native collagen fibers forming the meniscus. In some instances, the collagen fibers within the treatment scaffold 10 may be oriented generally parallel to the collagen fibers forming the meniscus, such that the collagen fibers within the treatment scaffold 10 are substantially aligned with the native collagen fibers forming the meniscus. In some cases, this can help with healing. In some cases, the treatment scaffold 10 may include loosely oriented type 1 collagen fibers with high porosity. It will be appreciated that the treatment scaffold 10, once disposed within the meniscal tear, may promote healing by providing a scaffold for biological material to fill up.

While not shown, the treatment scaffold 10 may include one or more biological factors, such as but not limited to substances that promote healing even in the absence of blood supply. These biological factors may be included particularly when the meniscal tear being treated is in the white zone of the meniscus, or sometimes even when the meniscal tear being treated is in the red-white zone of the meniscus. In some cases, repairs to the white zone of the meniscus do not heal well because of a lack of blood flow within the white zone of the meniscus. In some cases, repairs to the red-white zone of the meniscus also suffer from limited blood flow, even though the red-white zone can have at least some blood flow, especially relative to the white zone. It is thought that inclusion of a scaffold such as a collagen scaffold in treating meniscal tears may improve healing, even in the absence of adequate blood flow. Inclusion of biological factors may aid in healing.

In some cases, and as shown in FIG. 1B, the physician or other professional may be provided with a kit 12 that includes several different treatment scaffolds that may include or are made of collagen, including a collagen scaffold 10a, a collagen scaffold 10b and a collagen scaffold 10c. While described with respect to collagen, other materials are also contemplated. While the kit 12 is shown as including a total of three collagen scaffolds 10a, 10b and 10c, this is merely illustrative, as the kit 12 may include any number of collagen scaffolds. In some cases, the kit 12 may include several different collagen scaffolds of differing sizes, and may also include one or more larger collagen scaffolds that are intended to be cut down prior to use. In FIG. 1B, the collagen scaffold 10a is shown as being smaller than the collagen scaffold 10 shown in FIG. 1A. The collagen scaffold 10b is shown as having a larger width than the collagen scaffold 10a, but a shorter length. The collagen scaffold 10c is shown as having a length that is greater than the length L of the collagen scaffold 10, and perhaps a slightly larger width as well.

While drawn as rectilinear, it will be appreciated that this is merely illustrative, as the collagen scaffold 10, 10a, 10b and 10c may take any desired shape to best fit at a desired treatment site such as but not limited to a meniscal tear repair. In some cases, the collagen scaffold 10, 10a, 10b and 10c may be largely rectilinear, with rounded over corners, for example. In some cases, the collagen scaffold 10, 10a, 10b and 10c may be at least partially ovoid. The first primary surface 104 and the second primary surface 106 may be planar or curved. In some cases, the collagen scaffold 10, 10a, 10b and 10c may curve around a treatment site. These are just examples.

The collagen used to form the collagen scaffold 10, 10a, 10b and 10c may come from any of a variety of different sources. Collagen is a main structural protein in the extracellular matrix found in various connective tissues and thus can be obtained from various animals. For example, the collagen used to form the collagen scaffold 10, 10a, 10b and 10c may be bovine-based, i.e., from cows. In some cases, the collagen used to form the collagen scaffold 10, 10a, 10b and 10c may come from bovine tendon material. As an illustrative but non-limiting example, bovine Achilles tendon is digested down to highly purified collagen, and is reconstituted into a sheet by spinning the fibers around a mandrel. The collagen scaffold 10, 10a, 10b and 10c may be formed of collagen that has been obtained in other methods as well.

In some instances, the collagen scaffold 10, 10a, 10b and 10c may include a combination of collagen fibers or material and other biocompatible fibers or materials. For example, the collagen scaffold 10, 10a, 10b and 10c may include a combination of collagen fibers and bioabsorable polymer fibers, if desired.

While the collagen scaffold 10, 10a, 10b and 10c may be formed having a variety of different porosity levels (defined as relative amount of void space to solid material), in some cases the collagen scaffold 10, 10a, 10b and 10c may have a porosity of at least 50 percent or more, at least 60 percent or more, at least 70 percent or more, or at least 80 percent or more. In some cases, the scaffold 102 may have a porosity of 60 percent to 90 percent, 70 percent to 90 percent, 80 percent to 90 percent, or 85 percent to 90 percent.

Another way to define the collagen scaffold 10, 10a, 10b and 10c is in terms of average pore size. Pore size refers to a diameter of voids, or empty spaces, formed within the collagen scaffold 10, 10a, 10b and 10c. Average pore size, accordingly, refers to an average diameter of these voids. In some cases, the collagen scaffold 10, 10a, 10b and 10c may have an average pore size of 20 microns or greater, or 30 microns or greater, or 40 microns or greater, or 50 microns or greater, or 60 microns or greater, or 70 microns or greater, or 80 microns or greater, or 90 microns or greater. In some cases, the collagen scaffold 10, 10a, 10b and 10c may have an average pore size that is in a range of 100 microns to 500 microns, 100 microns to 400 microns, 100 microns to 300 microns, 100 microns to 150 microns, or 200 microns to 400 microns, for example.

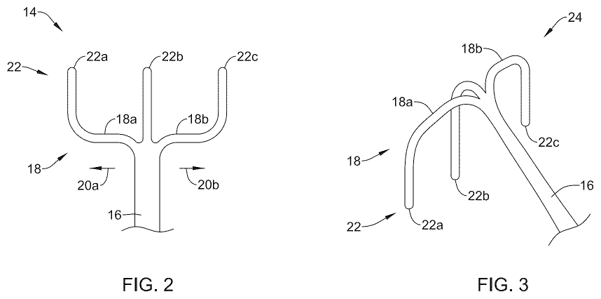

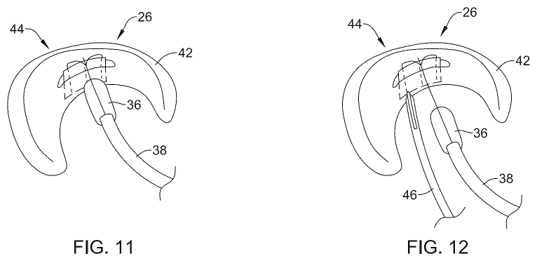

Once the collagen scaffold 10, 10a, 10b and 10c has been appropriately trimmed for use, or has been appropriately selected to have the appropriate size, the collagen scaffold 10, 10a, 10b and 10c can be delivered. The collagen scaffold 10, 10a, 10b and 10c may be delivered in a variety of ways. For example, the collagen scaffold 10, 10a, 10b and 10c may simply be delivered to a meniscal tear using a grasping device and optionally a standard cannula. In some cases, such as for radial tears and/or complex tear geometries, delivering the collagen scaffold 10, 10a, 10b and 10c with a graspers may be optimal. In some instances, the collagen scaffold 10, 10a, 10b and 10c may be delivered using a delivery device that includes a sheath that extends over the scaffold 10, 10a, 10b and 10c in order to hold the scaffold 10, 10a, 10b and 10c in a collapsed configuration for delivery. Once the scaffold 10, 10a, 10b and 10c has been delivered to a position proximate a meniscal tear, the scaffold 10, 10a, 10b and 10c may be deployed from the delivery device by retracting the sheath extending over the scaffold 10, 10a, 10b and 10c, or perhaps by pushing the scaffold 10, 10a, 10b and 10c distally out of the sheath. Other systems for delivering the scaffold 10, 10a, 10b and 10c are contemplated. In some cases, however, the collagen scaffold 10, 10a, 10b and 10c may be delivered using a delivery device such as that described with respect to FIGS. 2 through 14.

FIG. 2 shows an illustrative blank 14 that may be processed from a flat sheet or strip of Nitinol. In some cases, the blank 14 may be laser cut, stamped or machined, for example. Other cutting processes are contemplated. As can be seen, the blank 14 has been cut to include an elongate member 16, a cross member 18 that extends laterally from the elongate member 16, including a first cross member segment 18a that extends laterally from the elongate member 16 in a first direction indicated by an arrow 20a, and a second cross member segment 18b that extends laterally from the elongate member 16 in a second direction indicated by an arrow 20b. In some cases, as shown, the second direction is opposite the first direction. The blank 14 includes a number of forks 22, individually labeled as 22a, 22b and 22c. While a total of three forks 22 are shown, this is merely illustrative, as the blank 14 may include any number of forks 22, such as a total of two forks 22, or four or more forks 22. As formed, the blank 14 may be considered as being planar, with the elongate member 16, the cross member 18 and the forks 22 being disposed within a single plane that is parallel to the page.

FIG. 3 shows a securement member 24 of an implant delivery device that may be formed by shape-setting the blank 14. As shown in FIG. 3, the securement member 24 has been processed such that the forks 22 extend downward (in the illustrated orientation) from the cross member 18. In some cases, the forks 22 may be considered as extending within a plane that is orthogonal, or at least substantially orthogonal, to a plane that may be imagined as extending through the elongate member 16 and the cross member 18. In some cases, each of the forks 22 may be considered as being parallel with each of the other forks 22. In some cases, one or more of the forks 22 may lie either in front of or behind the imaginary plane extending through the forks 22 (and orthogonal or at least substantially orthogonal to the imaginary plane extending through the elongate member 16 and the cross member 18). The securement member 24 may be considered as having a remembered shape that matches that shown in FIG. 3. Even if the securement member 24 is moved out of the shape shown in FIG. 3, the securement member 24 will attempt to regain the shape shown in FIG. 3 anytime that the securement member 24 is not constrained from doing so.

The securement member 24 may have dimensions that are determined at least in part upon the final dimensions of the collagen scaffold 10, 10a, 10b and 10c. For example, the forks 22 may have a length that is at least as long as the width W of the collagen scaffold 10, 10a, 10b and 10c. The cross member 18 may have a length that is about as long as the length L of the collagen scaffold 10, 10a, 10b and 10c. If the collagen scaffold 10, 10a, 10b and 10c is particularly thin, this may mean that the forks 22 have a relatively smaller diameter, for example. In some cases, different size securement members 24 may be selected, based on the overall dimensions of the collagen scaffold 10, 10a, 10b and 10c.

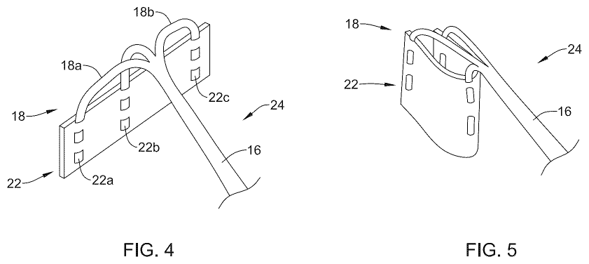

FIGS. 4 through 6 show an example of securing the collagen scaffold 10 (which could be any one of the collagen scaffolds 10, 10a, 10b and 10c described herein, or variations thereof) relative to the securement member 24 of an implant delivery device. For example, the forks 22 of the securement member 24 may be secured to the collagen scaffold 10. As shown in FIG. 4, in some instances the securement member 24 may be secured to the collagen scaffold 10 by penetrating the forks 22 into the collagen scaffold 10, such as by threading the forks 22 through the collagen scaffold 10. As shown, each of the forks 22 appear to have been threaded into the collagen scaffold 10, with the material forming the collagen scaffold 10 alternatively weaving in front of and behind each of the forks 22. This is merely illustrative, as in some cases, and depending on the particular dimensions of the collagen scaffold 10, each of the forks 22 may simply be inserted into spaces within the collagen scaffold 10, without threading the forks 22 into the collagen scaffold 10, or otherwise secured to the collagen scaffold 10.

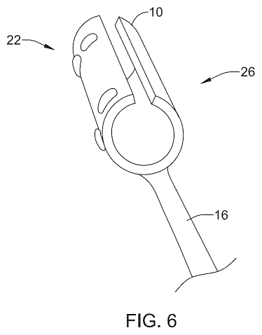

Once secured to the securement member 24 of the delivery device, the collagen scaffold 10, as well as the securement member 24 may be collapsed, or otherwise manipulated into a collapsed, delivery configuration. For example, in FIG. 5, the collagen scaffold 10 has been folded over on itself. This involves the cross members 18a and 18b each being adapted to fold over on themselves. In FIG. 6, and after being folded, the collagen scaffold 10 has been rolled up. This involves bending each of the forks 22. First folding, and then rolling up the collagen scaffold 10 allows the collagen scaffold 10 and the securement member 24 to, in combination, take up a reduced volume, which can help with disposing the collagen scaffold 10 and the securement member 24 in a delivery device, as will be discussed. It will be appreciated that bending the cross member 18 (in folding the collagen scaffold 10) and bending the forks 22 (in rolling up the folded collagen scaffold 10) cause the securement member 24 to be deformed from its remembered shape, or configuration. The folded and/or rolled up collagen scaffold 10, in combination with the similarly deformed securement member 24, may be considered as forming a scaffold assembly 26. In some cases, the scaffold assembly 26 may be held in this deformed, collapsed shape by virtue of a sheath or other structure temporarily placed over the scaffold assembly 26. In some cases, the scaffold assembly 26 may be assembled prior to a surgical procedure, such as subsequent to selecting an appropriately sized collagen scaffold 10, or after trimming a collagen scaffold to be an appropriate size. In some cases, the scaffold assembly 26 may be provided already assembled, and the physician or other professional merely needs to load the scaffold assembly 26 into a delivery device. In some cases, the physician or other professional may select a particular scaffold assembly 26 out of several different scaffold assemblies, where each of the several different scaffold assemblies include a particular size of collagen scaffold. This allows use of an appropriately sized collagen scaffold for the particular meniscal tear to be repaired. A first scaffold assembly 26 may include a small-sized collagen scaffold, a second scaffold assembly 26 may include a medium-sized collagen scaffold and a third scaffold assembly 26 may include a large-sized collagen scaffold, for example.

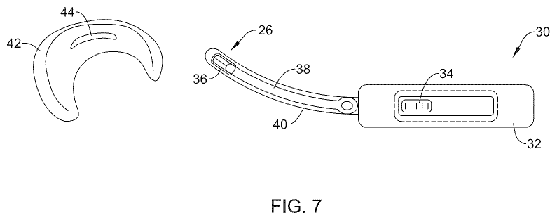

FIG. 7 schematically shows a delivery device 30 into which the scaffold assembly 26 has been loaded. The delivery device 30 includes a handle 32 including an actuation mechanism, such as a trigger 34 that is adapted to be movable back and forth within the handle 32. A sheath 36 is disposed over the scaffold assembly 26 in order to hold the scaffold assembly 26 in its current, collapsed configuration. In some cases, the scaffold assembly 26 would be provided already loaded into the delivery device 30. If the scaffold assembly 26 is to be cut to size before use, the physician would weave or spear the fork tines through the scaffold assembly 26, then roll the fork-scaffold assembly up, followed by advancing the trigger 34 to advance the sheath 36 over the rolled-up scaffold. In some cases, the sheath 36 may be clear or translucent, such that components within the sheath 36 may be visualized through the sidewall of the sheath 36. The delivery device 30 may also include an elongate shaft 38. The scaffold assembly 26, including the securement member 24 and the collagen scaffold 10 may be secured to the distal end of the elongate shaft 38. The elongate shaft 38 may be formed of any desired material, such as a malleable metallic material, for instance stainless steel or martensitic nitinol, for example.

In some cases, the elongate shaft 38 may initially be straight, and may be curved or otherwise bent in order to best reach a location of a particular meniscal tear within a particular patient. In some cases, the elongate shaft 38 may be adapted to be able to be manually bent by hand, which allows the physician or other professional to manipulate the elongate shaft 38 within their hands in order to bend the elongate shaft 38 to a desired curved shape during a surgical procedure. In some cases, the physician or other professional may utilize a mold in bending the elongate shaft 38 to a desired curved shape. In some cases, utilizing a mold may provide a smoother bend to the elongate shaft 38, with less chance of kinking the elongate shaft 38. US Pat. App. Pub. No. 2022/0161306, the disclosure of which is incorporated herein by reference, provides an example of a suitable mold.

A cannula 40 may extend over the elongate shaft 38. The cannula 40 may have a proximal end located proximate to the handle 32 and extend distally therefrom to a distal end of the cannula 40. The cannula 40 may extend distal of the elongate shaft 38 and cover the collapsed scaffold assembly 26 for delivery to an anatomical location.

In FIG. 7, the delivery device 30 is shown next to a schematically illustrated meniscus 42 that has a meniscus tear 44. It will be appreciated that the meniscus tear 44, as shown, is a vertical longitudinal tear. Other types of meniscus tears are known and contemplated herein.

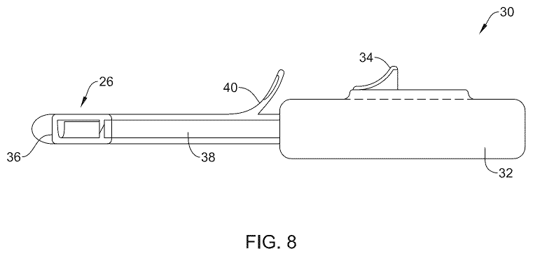

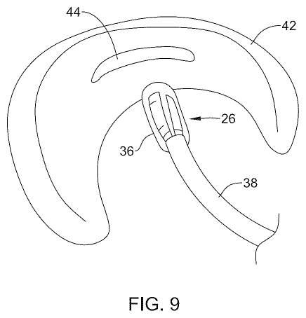

In FIG. 8, the cannula 40 can be seen as being partially removed. In some cases, the cannula 40 is a tear-away cannula that can simply be pealed away and disposed of. For example, a proximal end portion of the cannula 40 may be grasped by a user and pulled relative to the handle 32 and the elongate shaft 38, thereby removing the cannular from the delivery device 30. The cannula 40 may be removed once the delivery device 30 has entered the joint space near the meniscus 42. Thereafter, the delivery device 30 may be advanced in the joint space to navigate the scaffold assembly 26 to the location of the meniscus tear 44. This is shown in FIG. 9, where the distal end of the delivery device 30, including the scaffold assembly 26 and the sheath 36 surrounding the scaffold assembly 26, are positioned proximate the meniscus 42 and the meniscus tear 44.

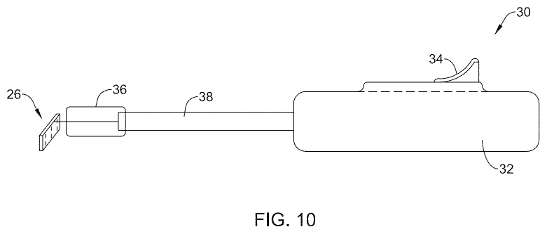

Once the scaffold assembly 26 has been positioned proximate the meniscus tear 44, the actuation mechanism provided with the handle 32 may be actuated to deploy the securement member 24 and the collagen scaffold 10 from the distal opening of the sheath 36. For example, as shown in FIG. 10, the trigger 34 has been moved proximally relative to the handle 32, thereby withdrawing the sheath 36 that was previously overlaying the scaffold assembly 26. As a result, the scaffold assembly 26, including the collagen scaffold 10 and the securement member 24, may be deployed out of the sheath 36 such that the securement member 24 may revert back to its remembered configuration in order to open or unfold the collagen scaffold 10 into a flattened configuration. The collagen scaffold 10 may then be inserted into the meniscus tear 44 in an expanded, flattened configuration. In some instances, the collagen fibers within the collagen scaffold 10 may be oriented generally parallel to the collagen fibers forming the meniscus such that the collagen fibers within the collagen scaffold 10 are generally aligned with the native collagen fibers forming the meniscus. In some instances, the collagen fibers within the collagen scaffold 10 may be oriented generally parallel to the collagen fibers forming the meniscus, such that the collagen fibers within the collagen scaffold 10 are substantially aligned with the native collagen fibers forming the meniscus.

In some cases, the securement member 24 may be used to hold the collagen scaffold 10 within the meniscus tear 44 as the meniscus tear 44 is repaired, such as by suturing the meniscus tear 44 closed with one or more sutures while retaining the collagen scaffold 10 within the meniscus tear 44. In other instances, the securement member may be withdrawn from the collagen scaffold 10 in order to get the securement member 24 out of the way prior to suturing. In some cases, as shown in FIG. 12, a secondary device 46 may be used to assist with the repair of the meniscal tear 44. For example a forceps or other grasping device may be used to grasp and/or manipulate the collagen scaffold 10 to correctly position and/or retain the collagen scaffold 10 within the meniscus tear 44. In some instances one or more of the sutures may extend through the collagen scaffold 10 as the sutures are sutured through meniscal tissue on both sides of the meniscus tear 44. In other instances, the sutures may not extend through the collagen scaffold 10 (e.g., the sutures may extend across the meniscus tear 44 above the collagen scaffold 10) as the sutured are sutured through the meniscal tissue on both sides of the meniscus tear 44.

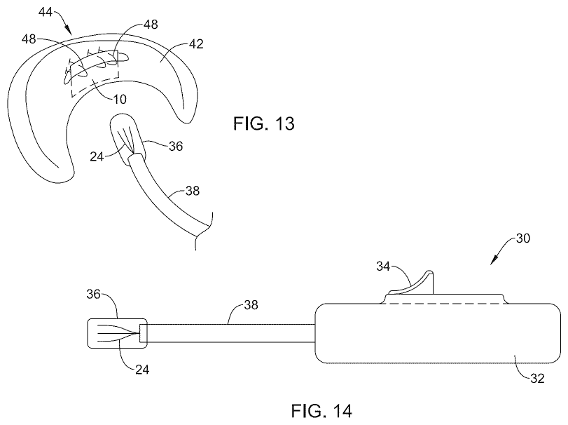

In some cases, the securement member 24 may be withdrawn back into the sheath 36 by actuating the actuation mechanism, such as moving the trigger 34 distally as shown in FIG. 14. This causes the sheath 36 to advance distally over the securement member 24, which protects the securement member 24 from contacting or damaging any anatomy during removal of the delivery device 30 from the joint space. FIG. 13 shows the repaired meniscal tear 44 with several sutures 48 that have been placed in order to secure the position of the collagen scaffold 10 within the meniscal tear 44. In some cases, the sutures 48 may be placed after the securement member 24 has been withdrawn from the collagen scaffold 10. In some cases, the sutures 48 may be placed while the securement member 24 remains within the collagen scaffold 10. The sutures 48 may be placed using any desired technique, process or equipment. In some instances, the sutures 48 may be passed through the thickness of the collagen scaffold 10 as the sutures are passed through the meniscal tissue to close the meniscal tear 44. While sutures are shown, other tissue fasteners are contemplated. In some cases, the sutures 48 may be placed using the FAST-FIT FLEX meniscus repair device available commercially from Smith & Nephew. In some cases, the secondary device 46 may represent the FAST-FIT FLEX meniscus repair device, for example. Additional suturing devices are disclosed in U.S. Pat. Nos. 7,887,551 and 8,986,343, the disclosures of which are herein incorporated by reference.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A method for repairing a meniscal tear within a patient's meniscus, the method comprising:
securing a meniscal implant relative to a delivery device to form a scaffold delivery assembly;
delivering the scaffold delivery assembly to a position proximate the meniscal tear;
inserting the meniscal implant into the meniscal tear; and
holding the meniscal implant within the meniscal tear by placing one or more fasteners extending across the meniscal tear.

2. The method of claim 1, further comprising separating the meniscal implant from the delivery device after inserting the meniscal implant into the meniscal tear.

3. The method of claim 2, wherein the method further comprises one of:
intra-operatively cutting the meniscal implant to an appropriate size prior to securing the meniscal implant to the delivery device; or
selecting an appropriately sized meniscal implant out of a plurality of differently sized meniscal implants.

4. The method of claim 1, wherein the delivery device comprises:
an elongate member;
a cross member including a first cross member segment extending laterally from the elongate member in a first direction and a second cross member segment extending laterally from the elongate member in a second direction; and
a plurality of fork members extending from the cross member, the plurality of fork members extending in a plane transverse to a plane extending through the elongate member and the cross member.

5. The method of claim 1, further comprising:
intra-operatively imparting a bend to an elongate shaft of the delivery device prior to delivering the scaffold delivery assembly to the position proximate the meniscal tear.

6. The method of claim 1, wherein the step of inserting the meniscal implant into the meniscal tear includes substantially aligning collagen fibers of the meniscal implant with collagen fibers of the meniscus.

7. A method for repairing a meniscal tear within a patient's meniscus, the method comprising:
inserting a meniscal implant secured to a distal end region of a delivery device into the meniscal tear; and
placing one or more fasteners extending across the meniscal tear through the meniscal implant to hold the meniscal implant within the meniscal tear;
thereafter, decoupling the delivery device from the meniscal implant.

8. The method of claim 7, wherein the delivery device comprises:
an elongate member;
a cross member including a first cross member segment extending laterally from the elongate member in a first direction and a second cross member segment extending laterally from the elongate member in a second direction; and
a plurality of fork members extending from the cross member, the plurality of fork members extending in a plane transverse to a plane extending through the elongate member and the cross member.

9. The method of claim 8, further comprising:
intra-operatively attaching the meniscal implant to the plurality of fork members prior to inserting the meniscal implant into the meniscal tear.

10. The method of claim 8, further comprising:
intra-operatively imparting a bend to the elongate shaft of the delivery device prior to inserting the meniscal implant into the meniscal tear.

11. The method of claim 7, wherein the step of inserting the meniscal implant into the meniscal tear includes substantially aligning collagen fibers of the meniscal implant with collagen fibers of the meniscus.

12. The method of claim 7, wherein the method further comprises one of:
intra-operatively cutting the meniscal implant to an appropriate size prior to inserting the meniscal implant into the meniscal tear; or
intra-operatively selecting an appropriately sized meniscal implant out of a plurality of differently sized meniscal implants prior to inserting the meniscal implant into the meniscal tear.

13. A method for repairing a meniscal tear within a patient's meniscus, the method comprising:
inserting a meniscal implant formed of collagen fibers into the meniscal tear such that the collagen fibers of the meniscal implant are substantially aligned with collagen fibers of the meniscus; and
placing one or more fasteners extending across the meniscal tear through the meniscal implant to hold the meniscal implant within the meniscal tear.

14. The method of claim 13, wherein the meniscal implant is inserted into the meniscal tear using a delivery device having the meniscal implant coupled to a distal end thereof.

15. The method of claim 14, wherein the delivery device comprises:
an elongate member;
a cross member including a first cross member segment extending laterally from the elongate member in a first direction and a second cross member segment extending laterally from the elongate member in a second direction; and
a plurality of fork members extending from the cross member, the plurality of fork members extending in a plane transverse to a plane extending through the elongate member and the cross member.

16. The method of claim 15, wherein the plurality of fork members include at least three forks.

17. The method of claim 15, further comprising:
intra-operatively attaching the meniscal implant to the plurality of fork members prior to inserting the meniscal implant into the meniscal tear.

18. The method of claim 14, further comprising:
intra-operatively imparting a bend to an elongate shaft of the delivery device prior to inserting the meniscal implant into the meniscal tear.

19. The method of claim 14, further comprising:
advancing the meniscal implant to the meniscal tear with the meniscal implant in a collapsed configuration within a sheath; and
unfolding the meniscal implant into a flattened configuration at the meniscal tear by deploying the meniscal implant from a distal end of the sheath.

20. The method of claim 13, wherein the method further comprises one of:
intra-operatively cutting the meniscal implant to an appropriate size prior to inserting the meniscal implant into the meniscal tear; or
intra-operatively selecting an appropriately sized meniscal implant out of a plurality of differently sized meniscal implants prior to inserting the meniscal implant into the meniscal tear.
